(19) United States
(12) Patent Application Publication
(10) Pub. No.: US 2024/0277342 A1
(43) Pub. Date: Aug. 22, 2024

(54) METHOD AND DEVICE FOR ANEURYSM REPAIR USING INTRAOPERATIVE ENDOLUMINAL COUPLING TO PRE-IMPLANTED STENT FOR DUAL BARRIER FORMATION

(71) Applicant: Carlos Mario Jimenez, Medellin (CO)

(72) Inventor: Carlos Mario Jimenez, Medellin (CO)

(21) Appl. No.: 18/583,889

(22) Filed: Feb. 22, 2024

Related U.S. Application Data
(60) Provisional application No. 63/447,348, filed on Feb. 22, 2023.

(51) Int. Cl. A61B 17/12; A61B 17/00

(52) U.S. Cl. CPC A61B 17/12145; A61B 17/12118; A61B 2017/00477; A61B 2017/00867

(57) ABSTRACT

The present invention consists of a flow diverter device deployed in the aneurysm neck to be used during an endovascular intervention. The device consists of a single filament that forms two spirals: the distal spiral (on the aneurysmal side) and the proximal spiral (on the vascular face of the stent). These spirals are continuous with a central spring that connects them. The spring passes through a predefined cell of a previously implanted stent. The distal and proximal barriers act as flow diverters. They cover a specific group of stent cells in front of the aneurysm neck. The intermediate spring traverses a stent cell, preventing movement. The two barriers, along with the connecting spring, play a crucial role in blocking any dislodgment after deployment. This invention combines precise positioning, flow diversion, and stability to address aneurysms effectively, avoiding direct manipulation of the lesion and respecting the parent vessel.

### STATE OF THE ART OF THE INVENTION

### Field of the Invention

The present invention pertains to the field of implantable medical devices and specifically introduces a device and a method applicable to closing focal defects in artery walls, including conditions like aneurysms.

These techniques fall within the domain of minimally invasive endovascular interventions.

Here are the key aspects of this invention:
Device purpose: The invention aims to address focal defects in artery walls, which can be critical in cases of aneurysms or other vascular issues.
Implantable barriers: The doctor who acts as an operator can implant two barriers within the lumen of a diseased artery. These barriers are assembled from a single filament. Their purpose is to provide a protective shield or closure for the affected area.
Integration with stent: The barriers are integrated by the operator and during an endovascular procedure onto a previously implanted stent within the same artery. This integration ensures precise positioning of the barriers at the focal defect site, such as the aneurysm neck. This concept was already exposed by the inventor who is responsible for this application in the provisional patent application before U.S. Pat. No. 6,344,7348, filed on Feb. 22, 2023.
Treatment precision: By achieving accurate positioning, the barriers allow for targeted treatment of focal defects in the vascular wall. This could significantly improve outcomes for patients with aneurysms or similar conditions.

This approach combines minimally invasive techniques, stent integration, and precise intraoperative barrier placement to enhance the treatment of vascular wall defects.

The following are other features of the device:
Intraoperative coupling: This device is connected during an endovascular intervention to an existing intravascular stent that was previously implanted. The operating physician performs this coupling intraoperatively, ensuring precise placement.
Single filament with memory shape: The device is assembled from a single filament with a memory shape. This memory shape allows it to return to a predetermined configuration even after deformation, ensuring stability and functionality.
Spirals and intermediate spring: The device comprises two spirals joined by an intermediate spring. These components likely serve multiple purposes, such as enhancing flexibility, adapting to vessel contours, and maintaining structural integrity.
Flow diverter function: When coupled to the struts of an existing stent in front of an aneurysm neck, the device acts as a flow diverter. Specifically, it creates a barrier along the neck of an aneurysm. By redirecting blood flow away from the weakened vessel wall, it promotes healing and prevents rupture.

In the context of aneurysms, this device diverts blood flow away only from the aneurysm's neck, respecting the parent blood vessel in its integrity, reducing the risk of rupture, and allowing the vessel wall to heal.

Arterial wall defects: Additionally, the device can address arterial wall defects resulting from medical procedures (e.g., arterial punctures during catheterization). Once coupled to the previously implanted intravascular stent, it facilitates repair by reinforcing the vessel wall.

Hemorrhagic defects: For patients with potentially hemorrhagic defects caused by catheter removal, this device provides a solution. By converting the conventional stent and device elements into a cohesive flow diverter, it aids in vessel wall repair.

In summary, this device—when coupled with an intravascular stent—offers a targeted approach to repairing defects within blood vessels. Its memory shape, spirals, and flow-diverting capabilities make it a valuable tool in endovascular procedures.

### DESCRIPTION OF THE PRIOR ART

Defects in the vascular wall: Basic concepts. Blood vessels are tubular organs arranged in an interconnected network and designed to transport blood to the different tissues of the human body; defects in the continuity of the vascular wall, as occurs in aneurysms, constitute lesions that can appear anywhere in the circulation, but are more frequently found in the aorta and in the cerebral circulation, and consist of deformation of the normal cylindrical wall of an artery, causing a specific segment to become a kind of blind sac, whose structure is weaker than that of the parent vessel and can eventually rupture and lead to life-threatening hemorrhage. The initial histopathological event in aneurysms formation is the disruption of the internal elastic membrane, which yields to damaging by hemodynamic forces, usually at arterial bifurcation sites, in which hemodynamic stress is high; progressively the layers of the arterial wall undergo severe degenerative changes that cause the aneurysm wall to be weak, lacking of smooth muscle cells in some areas in the middle layer, which is the area that gives an arterial vessel structural integrity. It is this structural weakness that potentially leads to rupture of aneurysms. Pereira V M 1, Brina O, Gonzalez A M, Narata A P, Ouared R, Karl-Olof L. Biology and hemodynamics of aneurysmal vasculopathies. Eur J Radiol. 2013 October; 82(10):1606-17. doi: 10.1016/j.ejrad.2012.12.012. Epub 2013 Jan. 21. The area of the vascular wall where the histological changes induced by hemodynamic forces begin and the saccular lesion appears is known as the neck of the aneurysm; once the saccular dilation is formed, it is through the neck that blood flow enters the aneurysm cavity, impacts against its wall and often ends up with its rupture. This is why any type of intervention aimed at preventing the rupture of an aneurysm must focus on the imposition of a mechanical obstacle, either by contact of the blood flow with the wall of the lesion, or by passing through its neck, since the absence of flow through this neck, or its extremely restricted passage, protects the aneurysmal wall from the harmful mechanical effects of blood flow, and eventually leads to thrombosis and closure of the lesion, eliminating the risk of rupture.
Intravascular stents without a covered segment: these are a group of devices designed to impose a mechanical obstacle that partially blocks the migration of some solid or liquid element from the defective wall of an artery, that is, from the lesion subjected to treatment. For this reason, in the case of focal lesions of the arterial wall, such as aneurysms, they are usually used more as a complementary device than as sole therapy. They have the advantage of their low metal content, with a very high porosity, their excellent navigability and good adaptability and tolerance to their presence by arterial vessels. Nakasaki M, Nonaka T, Nomura T et al: Cerebral aneurysm neck diameter is an independent predictor of progressive occlusion after stent-assisted coiling. Acta neurochir (Wien) 2017 May 9. doi: 10.1007/s00701-017-3199-8. [Epub ahead of print]—Lubicz B, Morais R, Bruyère P J, Ligot N, Mine B: Stent-assisted coiling of wide-neck bifurcation aneurysms with a branch incorporated in the aneurysm base: long-term follow-up in 49 patients with 53 aneurysms. Neuroradiology 2017 Apr. 11. doi: 10.1007/s00234-017-1834-y. [Epub ahead of print]
Covered stents: they are designed to correct a focal defect in the wall of an artery, and what is pursued with them is to hinder the contact of the blood flow with that defect. In them it is not possible to position a covered segment in front of the focal defect, and therefore the non-porous covered area of the stent can cover segments of the vessel from which branches originate, which if occluded can cause ischemic problems.
Partially covered stents: In the case of stents with only a covered segment, it is almost impossible for the operator to adequately position the covered area in front of the lesion, respecting the rest of the arterial wall, since the operator would have to manipulate the stent, making it rotate on its own axis, to achieve a adequate positioning of the covered segment both in the coronal, sagittal and azimuthal planes. It is well known to those skilled in the art that such manipulation can cause severe endothelial damage, as well as spastic reaction of the arterial wall.
Flow diversion devices: they are devices with low porosity, with which it is sought to divert the blood flow, in such a way that it does not contact the site of the focal defect that is being treated. They have the drawback of the high number of wires to make their pores, and therefore the high metal mass of their composition, which can trigger intimal hyperplasia, and also the eventual occlusion of the vessels adjacent to the defect, which can cause undesirable ischemic problems.

### COMPARISON OF THE PRESENT INVENTION WITH THE PREVIOUS STATE OF THE ART

Researchers continue to explore novel techniques and approaches to improve aneurysms treatment strategies, considering both the lumen (inside the vessel) and the wall of the artery. The goal is to achieve effective treatment while minimizing contact with delicate vessel walls. There is a growing need for an improved method of treatment of focal defects in the arterial wall, such as aneurysms, which provides a seal of the neck of the lesion, respecting the rest of the vessel and without the need to contact the delicate walls of the aneurysmal cavity, which carries a relatively high risk of iatrogenic rupture. In this way, tissue regeneration can be achieved, leading to a permanent repair, and where the seal will not be subjected to recanalization, with the consequent persistence of the aneurysm.
Covered stents indeed pose unique challenges due to their rigidity. However, they offer benefits such as promoting the occlusion of adjacent branches and potentially preventing thrombosis of the arterial lumen. Here are some of the difficulties with covered stents:

Navigation difficulties: covered stents can be challenging to navigate due to their stiffness. Their rigid structure may hinder precise placement within the vessel.

Interventionists must carefully maneuver these stents to ensure optimal coverage of the target area while avoiding damage to surrounding tissues.

Branch occlusion: covered stents can inadvertently occlude nearby branches or side vessels. This occurs when the stent extends beyond the intended treatment site. While this can be advantageous for aneurysms or other vascular defects, it requires careful planning to minimize unintended blockage of critical vessels.

Intimal hyperplasia: refers to the thickening of the inner lining (intima) of blood vessels. It can occur in response to stent placement. Covered stents may trigger slow, progressive intimal hyperplasia. This thickening can narrow the arterial lumen over time, potentially leading to reduced blood flow or even thrombosis.

Clinicians must weigh the advantages of aneurysm exclusion and branch protection against the potential drawbacks of covered stents. Close monitoring and follow-up imaging are crucial to detect any adverse effects promptly. (Romaguera R, Waksman R. Covered stents for coronary perforations: is there enough evidence? Catheter Cardiovasc Interv. 2011 Aug. 1; 78(2):246-53. doi: 10.1002/ccd.23017. Epub 2011 Jul. 15. PMID: 21766425)

### The Advantages of this Invention Compared to Covered Stents

Navigation simplicity: the device's single, highly navigable filament minimizes navigation challenges. When the carrying microcatheter is retracted, the filament naturally reconstitutes its shape, streamlining the process.

Non-interference with parent vessel: unlike covered stents, this invention does not interact with the parent vessel. This crucial feature helps prevent issues like neointimal hyperplasia and occlusion of critical branches.
Flow-diverter stents (FDS): neo-intimal hyperplasia (NIH) is a common occurrence after the implantation of flow-diverter stents (FDS). Although it is often asymptomatic, this vascular response can occasionally lead to delayed ischemic strokes. FDS are frequently accompanied by neo-intimal hyperplasia, given their high metal content, usually around 30%. Likewise, they can cause thrombosis of the vessels adjacent to the focal defect that is being corrected. (Sindeev S, Prothmann S, Frolov S, Zimmer C, Liepsch D, Berg P, Kirschke J S, Friedrich B. Intimal Hyperplasia After Aneurysm Treatment by Flow Diversion. World Neurosurg. 2019 February; 122:e577-e583. doi: 10.1016/j.wneu.2018.10.107. Epub 2018 Oct. 26. PMID: 31108073).

NIH is a dual-vessel reaction after FDS implantation. When planning treatment in locations at risk of ischemic complications due to severe NIH, stent design should be considered. Minimal NIH might also be necessary, as it plays a role in aneurysm healing. Patients should receive recommendations for optimal medical management of their cardiovascular risk factors before treatment to prevent excessive NIH reactions.

The advantages of this invention compared to flow diverter stents:
Navigation simplicity: again, the device's single, highly navigable filament minimizes navigation challenges. When the carrying microcatheter is retracted, the filament naturally reconstitutes its shape, streamlining the process.
Non-interference with parent vessel: unlike flow diverter stents, this invention does not interact with the parent vessel. This crucial feature helps prevent issues like neointimal hyperplasia and occlusion of critical branches.
The following concepts highlight the advantages of the method and the device described in the present invention in comparison with the previously used methods and devices:

This invention allows the operating physician to treat focal defects in the wall of an artery, by implanting two barriers exclusively in front of the focal lesion, without affecting the rest of the artery and without manipulating the device in contact both with the normal vessel and the diseased wall (for example, an aneurysm), thus preventing intraoperative rupture of the vessel, occlusion and thrombosis of the parent artery, as well as the branches adjacent to the focal lesion. The method is based on taking advantage of the prior implantation of a bare conventional intravascular stent, with low metal content and high porosity, that is easily navigable and of proven tolerance by patients, so that the operating physician can adjoin the two barriers against both sides of said stent, during the course of the endovascular intervention, affecting only the focus of the lesion being treated (for example in front of the neck of an aneurysm), without compromising the non-diseased areas of the arterial wall or sacrificing the origin of the arterial branches adjacent to the focal lesion.

The potential advantages of this invention compared to the method of a single-layer stent with uniform porosity in the parent vessel for the treatment of the focal defect include: a high metal-to-artery ratio is not imposed and put in contact with the vascular endothelium, with the risk of reactive intimal hyperplasia and stenosis or even thrombosis of the vessel into which the stent is implanted. In addition, the risk of occlusion of the ostium or origin of vessels adjacent to the neck of the aneurysm is eliminated.

The potential advantages of this invention compared to the method of deploying a stent in the parent vessel, where this stent has pre-formed areas of different porosity, include again, no high metal-to-artery ratio is imposed on the vascular endothelium, thereby triggering the risk of intimal hyperplasia and stenosis or even thrombosis of the vessel into which the stent is deployed. In addition, the risk of occlusion of the ostium or origin of vessels adjacent to the focal defect in the vascular wall, for example an aneurysm, is eliminated.

Potential advantages of this invention compared to the method of deploying a stent in the parent vessel, where this stent has areas of different porosity that are created by modifying the stent surface after its expansion include: again, a high metal-to-artery ratio is not imposed on the vascular endothelium, with the risk of intimal hyperplasia and stenosis or even thrombosis of the vessel into which the stent is deployed. In addition, it is eliminated the risk of occlusion of the ostium or origin of the vessels adjacent to the focal wall defect, such as an aneurysm.

Potential advantages of this invention compared to the method of deploying a stent in the parent vessel as a radial expansion element and which is accompanied by a lateral expanding occluding element, which is independent of the stent include: with the device and the method presented in this invention, it is not necessary to implant an occluding element inside the focal defect of the vascular wall, for example in aneurysms, but rather everything is reduced to the implantation of a device coupled to a stent previously implanted in the vascular lumen. In this way, the need to contact the walls of the focal defect and to insert an element in contact with them is eliminated, leaving aside the risk of iatrogenic rupture of the arterial focal lesion.

Advantages of the method and device described in the present invention compared to previously used methods and devices for treating aneurysms:
Selective Treatment: The invention allows the operating physician to precisely address focal defects in the artery wall. By implanting two barriers exclusively in front of the focal lesion, the rest of the artery remains unaffected. Importantly, this approach avoids manipulation of the device in contact with both the normal vessel and the diseased wall (e.g., an aneurysm). As a result, intraoperative rupture of the aneurysm or the vessel, occlusion, and thrombosis of the parent artery, as well as branches adjacent to the focal lesion, are prevented.
Leveraging prior stent implantation: the method capitalizes on the prior implantation of a bare conventional intravascular stent. This stent has low metal content and high porosity, making it easily navigable and well-tolerated by patients. During endovascular intervention, the operating physician can position the two barriers against both sides of the existing stent. This targeted approach affects only the focus of the lesion being treated (e.g., the neck of an aneurysm). Importantly, non-diseased areas of the arterial wall and the origin of arterial branches adjacent to the focal lesion remain uncompromised.
Advantages over single-layer stents: compared to the method of using a single-layer stent with uniform porosity in the parent vessel for treating focal defects, this invention avoids imposing a high metal-to-artery ratio in direct contact with the vascular endothelium. This mitigates the risk of reactive intimal hyperplasia, stenosis, or even thrombosis of the vessel where the stent is implanted. Furthermore, the risk of occlusion at the ostium or origin of vessels adjacent to the neck of the aneurysm is eliminated.

In summary, this approach offers targeted treatment while minimizing adverse effects on healthy vessel segments and adjacent branches, with the following additional advantages:
Precise targeting: the device allows the operating physician to focus exclusively on the focal lesion (e.g., the aneurysm neck) without disturbing the rest of the artery. By implanting two barriers in front of the lesion, the non-diseased areas of the arterial wall remain untouched. This precision prevents intraoperative rupture of the aneurysm or the vessel, a critical concern during such procedures.
   Preserving adjacent vessels: unlike traditional methods, which may affect adjacent vessels or branches, this approach spares the origin of arterial branches near the focal lesion. The risk of occlusion or thrombosis in these adjacent vessels is minimized.
Avoiding high metal-to-artery ratio: the device builds upon a prior implantation of a bare conventional intravascular stent. This stent has low metal content and high porosity, reducing the risk of reactive intimal hyperplasia and stenosis. By avoiding direct contact with the vascular endothelium, the risk of vessel complications is significantly lowered.

In summary, this method provides a safer and more precise way to address aneurysms, enhancing patient outcomes while minimizing risks associated with traditional approaches.

### BRIEF DESCRIPTION OF THE INVENTION

The objective of this invention is to address focal defects in artery walls, specifically those associated with aneurysms. The device comprises elements that are incorporated during an endovascular intervention into an existing high-porosity stent positioned in front of the aneurysm neck. These elements act as a double barrier, diverting blood flow within a specific segment of the stent, in the aneurysm neck area, effectively isolating the lesion from direct contact with the blood flow. The barriers are strategically placed on both sides of a preselected segment of the stent. By doing so, the device prevents blood flow from directly interacting with the aneurysm neck. Notably, when the inflow through the aneurysm neck is restricted by 40% or more, a beneficial effect occurs over time: lesion regression and thrombosis. This approach aims to promote healing and reduce the risk associated with aneurysms. By isolating the lesion, the device encourages natural processes that lead to lesion closure.

These circular barriers are assembled from a single filament, forming a spiral shape. A spring connects the two circular barriers. This intermediate spring continues from the same filament used for the barriers. The spring crosses a stent cell, integrating with the existing stent. The device serves as a double barrier within the stent. Said spring also prevents blood flow from detaching the device and dragging it downstream. By positioning the barriers on either side of the stent, it effectively isolates the aneurysm neck from direct blood flow. The device is navigable and implantable using endovascular methods. Importantly, it is fully retrievable before final deployment, aligning with the operator's intent. Radiopaque markers aid in locating, positioning, and monitoring the device during and after deployment. While this description provides exemplary embodiments, it acknowledges that various components and structures can be combined to yield enhanced functionality. Proficient operators can integrate different elements to create further devices.

The flexibility of this invention allows the operator to make precise decisions during the intervention. By choosing the optimal site for implanting the two barriers, they can align with the axial, sagittal, coronal, and azimuthal planes of the focal lesion within the arterial wall. This adaptability ensures a tailored approach to treating the defect, enhancing patient outcomes.

Regarding the elements that constitute the device, the distal barrier is positioned at the far end, closest to the target site; it acts as the initial barrier to divert blood flow and is composed of the same filament material. The central spring connects the distal and proximal barriers, functions as a flexible bridge, and continues from the same filament, maintaining structural integrity. The proximal barrier is located at the proximal end from the target site. It completes the double barrier system, and it is also part of the continuous filament. The use of biocompatible and shape-memory materials ensures compatibility with the body and allows the device to adapt to its intended purpose.

The device components are initially constrained within a catheter. During the intervention, they are expanded and released by the operator. Endovascular techniques are followed, ensuring safe and precise placement. The device is coupled to a previously implanted stent. It passes through one of the stent cells, located at the aneurysm neck. The filament becomes exposed when the delivery catheter is retracted. The distal barrier forms on the aneurysmal side, followed by the central spring through the cell, and finally the proximal barrier on the vascular side of the stent. The barriers meet the stent struts, maintaining a specific separation distance (at least, 2 mm). The central spring elongates when loaded into the catheter.

Upon retraction of the catheter, the components are exposed, and the intermediate spring brings the barriers closer together. The device mates with the preselected stent cell, separated only by the stent struts. A detachment mechanism (mechanical or electrothermal) is positioned at the proximal end of the device. If the detachment mechanism is not activated, the entire device can be retrieved. It can be packed into the microcatheter that delivered it to the patient's circulation. The device is transportable within a 1.5 French or larger microcatheter.

On the other hand, shape memory alloys (SMAs), also known as memory metals, memory alloys, smart metals, or muscle wires, exhibit an outstanding behavior: they allow deformation when cold but return to their original shape when heated. The core concept to this behavior lies in the phase transformation that occurs within SMAs. The two most prevalent SMAs are copper-aluminium-nickel and nickel-titanium (NiTi) alloys. When cooled, these alloys transform from the high-temperature phase (called austenite) to the low-temperature phase (called martensite). Heating them reverses this transformation.

This property is widely exploited in the manufacture of medical devices and is well known to those skilled in the endovascular art. SMAs additionally exhibit an additional property called superelasticity, allowing them to absorb and recover large deformations without permanent damage. While other SMAs exist (like iron-based and copper-based ones), NiTi-based SMAs are preferred for most applications due to their stability and superior thermo-mechanical performance. NiTi alloys undergo phase transformations at specific temperatures, making them reliable and practical. This is the preferred material for the present invention.

The conformation of the device has the following purpose:

The design of the device ensures that the occluding components activate their shape-memory once the microcatheter containing them is retracted. Upon deployment, they effectively fulfill their purpose by occluding or isolating the defect in the vascular wall, such as the neck of an aneurysm. This approach contributes to safer and more precise interventions.

The barriers have a dual purpose, as they obstruct blood flow, preventing it from passing through, and, at the same time, they are a coupling mechanism, when placed against and adjoined to a preset segment of the stent. This dual functionality ensures stability and prevents migration downstream. When released by the operator during an endovascular procedure, the pre-shaped filament conforms a pair of circular barriers, connected by an intermediate spring. They are secured on both sides of the stent, and their diameter becomes larger than that of the stent cell. In this way, they remain blocked by the stent struts, maintaining their position.

The spring, a resilient intermediary, bridges the two barriers. Its purpose is to regain its original shape upon deployment, contracting in length. As it does so, it ensures that the proximal barrier deploys near the already deployed distal barrier, which is crucial for the device's effectiveness. By maintaining this strategic arrangement, the barriers work harmoniously to achieve their intended purpose within the arterial system—flow diversion restricted to the aneurysm neck. Their strategic placement—against and adjoined to each side of the stent—ensures separation only by several sturdy struts.

The pusher wire, a familiar tool for skilled endovascular practitioners, plays a crucial role in navigating implantable devices within blood vessels. This pusher wire allows precise movement of the device to the specific location within the vascular wall where the defect (such as an aneurysm neck) exists. The proximal end of the pusher wire remains outside the patient and is controlled by the operator. The distal end of the pusher wire is in direct contact with the detachment mechanism of the device. As the operator manipulates the pusher wire, it translates their intentions to the device. The pusher wire ensures that the device is accurately positioned for optimal treatment.

### BRIEF DESCRIPTION OF THE DRAWINGS

To illustrate the different steps of the process necessary for the present invention to fulfill its objective, the following figures are presented:

FIG. 1 shows the device with all its components, implanted through stent coupling, in front of the neck of an aneurysm.

FIG. 2 shows the intermediate spring between the distal and proximal spirals. Notice how this spring acquires a tubular shape, to complete the path formed by the channel of a stent cell.

FIG. 3a shows the distal spiral and only the hint of the proximal spiral, once the intermediate spring connecting the two spirals, distal and proximal, has been deployed.

FIG. 3b illustrates the distal spiral already fully deployed, and the proximal spiral deployed at about 50% of its final size.

FIG. 3c shows the two spirals, distal and proximal, fully deployed and conformed, as they should be at the end of the procedure.

FIG. 4 is an illustration of the device in its entirety, now fully deployed.

FIG. 5 illustrates how the thickness of the struts that make up a stent cell corresponds to the distance of the distal and proximal components of the device.

FIG. 6 presents a panoramic view of the device, showing the distal and proximal spirals, adopting a convex conformation once deployed (due to the tubular shape of the stent in the parent vessel on which they rest), as well as the intermediate spring that joins these spirals.

FIG. 7 shows the first step that the operating physician must carry out, which is the navigation of a catheter until its end is positioned in a pre-selected cell of the stent, until it is slightly exceeded.

### DETAILED DESCRIPTION OF THE INVENTION

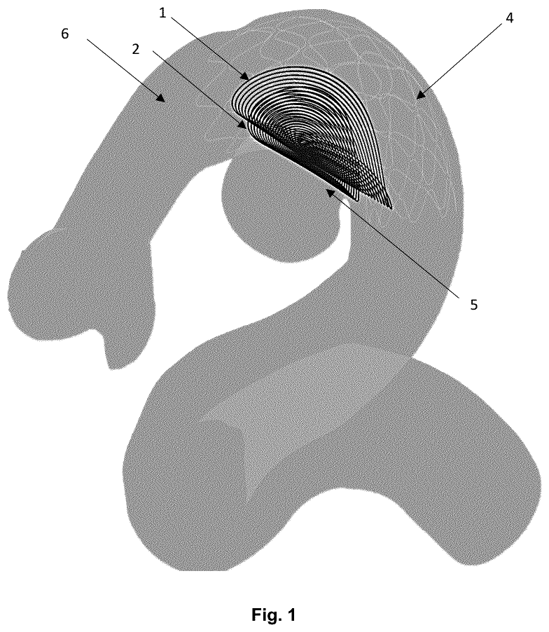

Taking the figures as a reference, the invention aims to correct vascular lesions, particularly aneurysms. It achieves this by occluding or isolating the lesion within the vascular wall. The device couples to a previously implanted stent 4, which is positioned in the parent vessel 6 in front of the aneurysm neck 5, as illustrated in FIG. 1. The device consists of two barriers that appose to the stent struts 7, when deployed by the specialist who performs the intervention, during an endovascular procedure. These barriers effectively divert blood flow to the lesion. By strategically placing the barriers against the stent, the device prevents migration or detachment. It ensures that the aneurysm neck is effectively isolated from blood flow.

The device is constructed from a single filament, that is shaped to create various components:
Two circular barriers 1, 2, that serve as occluding elements. Their purpose is to isolate the aneurysm neck from blood circulation.
Intermediate spring 3: connects the distal 2 and proximal 1 barriers. It ensures flexibility and structural integrity of the device, and when deployed, the spring contracts, ensuring the rapprochement (proximity) of the two barriers, a crucial step for the device's effectiveness.

By positioning the two barriers against each side of the stent, they work harmoniously to achieve their intended purpose within the parent vessel. The device is positioned against each side of a conventional intravascular stent 4, targeting a preset segment of the stent. The barriers effectively divert the inflow jet to the aneurysm. The spring is a continuous component, seamlessly connected to both the distal and proximal circular barriers. It ensures flexibility and structural integrity.

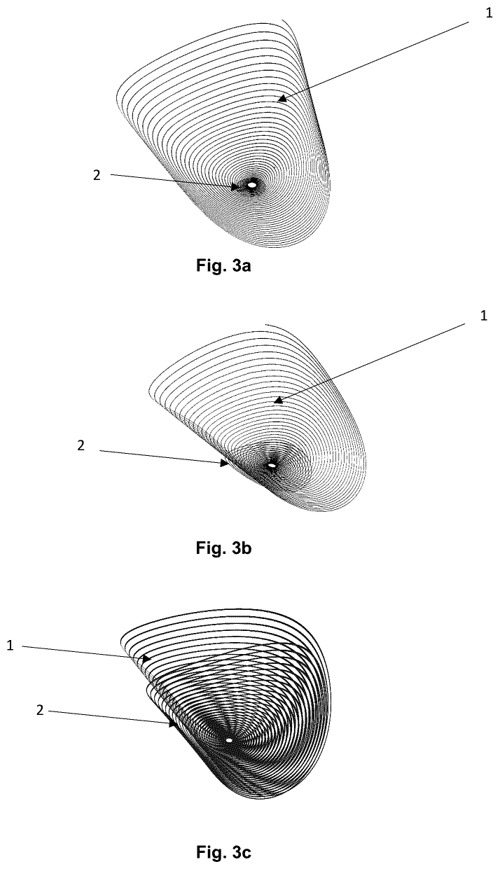

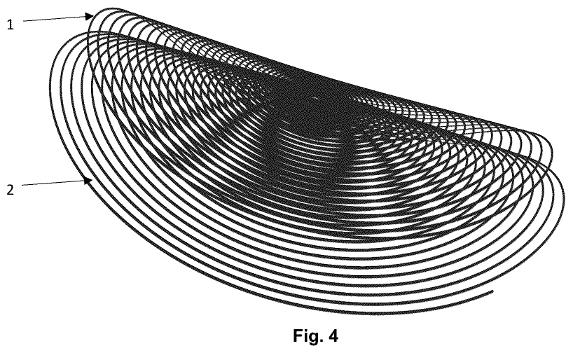

The device is initially transported in a constrained state within a catheter. Endovascular techniques are employed for precise navigation. Once positioned in front of a defect in the vascular wall (such as an aneurysm neck), it is expanded FIG. 3a, 3b, 3c. Almost simultaneously, it attaches to the pre-implanted stent. Finally, the device is released. If the device has not been fully released, it remains fully retrievable, and the operator can retrieve it following their intent. The device serves as an occluding endovascular system, which couples to the struts of a previously selected group of stent cells. The occluding elements consist of two barriers: distal 2 and proximal 1. These barriers achieve their objective from the vascular lumen and in front of the lesion. The operator does not need to enter the interior of the lesion, as the entire process occurs from within the lumen of the diseased vessel.

The device is manufactured from a single filament, The manufacturing process for this device relies on the remarkable properties of nitinol—a metal alloy composed of nickel and titanium, which is considered the preferred material for the manufacturing process of this device. Nitinol combines nickel (Ni) and titanium (Ti) in specific proportions, and this alloy exhibits a unique property called shape memory effect: when deformed (e.g., compressed or bent), it can return to its original shape upon heating. This behavior is crucial for the functionality of implantable devices. Nitinol also demonstrates superelastic behavior: It can undergo significant deformation without permanent damage. Upon unloading, it reverts to its original shape, and this property is ideal for medical devices that need flexibility and resilience. Nitinol is preferred for manufacturing various implantable devices. Examples include stents, guidewires, and occlusion devices (like the one described here). Its biocompatibility and mechanical properties make it suitable for use within the human body. In the intricate world of medical materials, nitinol plays a vital role in enhancing patient care.

One or more of the following materials may also be used: stainless steel, cobalt-chromium alloy, titanium or a titanium alloy, tantalum or a tantalum alloy, polymer-based resin or other polymer; it can also be manufactured from various combinations of these alloys. It is well known to those with experience and skill in the art that shape-memory alloys are a special class, as they have a property that makes them superelastic and allows them to “remember” a preselected shape during their manufacturing process, once they are made and deployed from within the catheter that transported them to their destination. Different polymers could also be used in some of the embodiments of this invention, including biodegradable polymers, such as those described below, for purposes of example, but not limited thereto: copolymers of polyglycolic acid or polylactic acid, poly D-lactide, poly D-L lactide, or copolymers obtained from combinations thereof. The occluding meshes (4), which adheres to the metallic structural framework (5), can be made of a material that provides the properties sought in the present invention, and can be selected from a group consisting of: a mesh; a network; a meshed; a membrane; a textile layer; a layer of a shape-memory material; a substantially flat patch of a compressible material; a component obtained from collagen extracellular matrix. As examples of materials for its manufacture, the following list is presented (although the specific material may not be limited to it): a metal alloy, a biocompatible textile material, such as a polymer, among them, but not limited to this list: polycarbonate polyurethanes, polyester polyurethanes, polyether polyurethanes, polysiloxone polyurethanes, or polyurethanes combined with soft elements, polyvinyl alcohols, polystyrene, nylon, polytetrafluoroethylene (PTFE), dacron, polyvinyl acetate, cellulose acetate, ethylene-propylene-diene monomer (EPDM), latex, silicone, polytetrafluoroethylene (PFTE), polyvinyl chloride, polyethylene, polyethylene ethafoam, polyethylene zotefoam. It may also be made from a bioabsorbable polymer, such as copolymers of polyglycolic acid or polylactic acid, poly D-lactide, poly D-L lactide, or copolymers obtained from their combinations, or similar. It can also be formed from the combination of two or more polymers, or from a polymer and a non-polymer. Another material that can be used is an elastomeric polymer, such as expanded polytetrafluoroethylene.

The design of this device, with its variable porosity, demonstrates a thoughtful approach to addressing vascular lesions. Let's highlight the key points:
The barriers (distal and proximal) exhibit varying porosity, as the central zone has very low porosity, and such porosity increases progressively toward the periphery. This design balances occlusion and blood flow. The device aims to occlude or isolate defects in the vascular wall (e.g., aneurysm necks), achieving this by strategically positioning the barriers, in such a way that allow blood flow to escape from the focal lesion (aneurysm), while they reduce blood flow into the lesion after deployment. This asymmetric porosity is advantageous over uniform porosity seen in other devices. In this sense, this invention aims to harmonize form and function.

Due to this same low porosity in the area directly related to the focal lesion in the arterial wall, such as the neck of an aneurysm 5, the two barriers do not allow gaps or channels through which blood flow enters the interior of the lesion with high velocity, which occurs with other prior art methods (such as coils and the stent-coil combination), and this is the most recognized explanation for the recanalization of the defect in the vascular wall, one of the problems presented with the prior art, which can be significantly reduced or completely avoided by means of the device and method presented in this invention. The micro-slits of the barriers are generated through processes such as etching, masking, the use of lasers or water jet cutting. The filament may be of uniform or non-uniform thickness, ranging from 0.005 inches (0.127 mm) to 0.050 inches (1.27 mm); preferably the thickness of the filament will range from 0.015 inches (0.381 mm) to 0.030 inches (0.762 mm). It will generally have a substantial degree of flexibility, elasticity, or resiliency that allows it and the device to be compressed within the delivery or navigation catheter and expanded adjoining the stent surface without the need to enter the focal lesion, and without causing damage to the vascular wall. The two barriers are substantially flat after its expansion, and, once the microcatheter is retracted and the filament is released they acquire their memory-shape to occupy a plane parallel to the axial longitudinal axis of the parent vessel, that is, the vessel that originates the aneurysm and on which a stent had previously been deployed.

Incorporating radiopaque markings into the device allows for effective monitoring during transport, deployment, and release. These markers enhance visibility using X-rays. Additionally, coating or plating the device with radiopaque substances (such as tungsten, tantalum, gold, or platinum) ensures clear visualization. The integration of radiopaque dot markers using various methods further enhances its functionality.

The device presented here is manufactured from a filament. In the manufacturing process, said filament must be given the shape of a double spiral 1, 2, both spirals joined by a central spring 3. The spirals formed by the filament, whether straight or given specific shapes, play a crucial role in achieving the desired stability and occlusive capacity. The distal spiral 2, which is the first to be deployed, has a circular shape, and its deployment begins with the largest peripheral loop, and progressively the other loops are formed, each time of smaller diameter, until it reaches the diameter of the distal loop corresponding to the spring or intermediate component. All the loops of this distal spiral are formed in the same plane, and therefore the distal spiral ends up being an essentially flat structure.

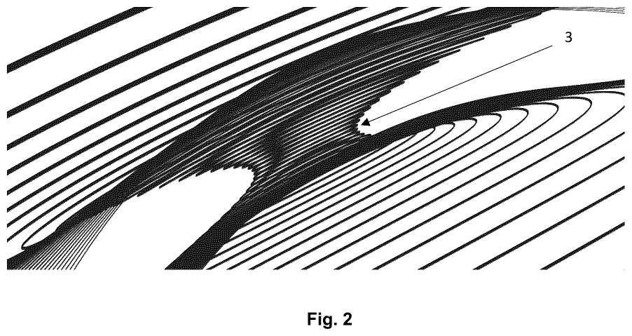

On the other hand, the central component of the device-referred to as the intermediate spring 3—plays a crucial role. The loops within the intermediate spring share the same diameter. However, they deploy in distinct planes, extending from distal to proximal; this arrangement results in a tubular structure, providing stability and flexibility. See FIG. 2. The most proximal loop of the intermediate spring seamlessly transitions into the central loop of the proximal spiral. The proximal spiral consists of loops that gradually increase in diameter as they progress toward the periphery. Both in the proximal 1 and distal 2 components, all the loops lie in the same plane. This arrangement creates an essentially flat structure, optimizing its function.

At the most proximal end of the outermost loop (the peripheral loop), two essential components converge: filament and pusher wire junction, and the detachment mechanism. This connects to the micro guidewire pusher, facilitating precise navigation. This detachment mechanism, well-known to seasoned endovascular practitioners, can be either mechanical or thermoelectric.

In relation to the method of use of the device, everything can be condensed into a widely used concept in the endovascular art, such as the deployment of a filament navigated within a microcatheter. This filament, when stripped by the retraction of the microcatheter that contains it, recovers the memory shape with which it was manufactured, to reconstitute itself as a structure that is made up of two circular spirals of approximately the same diameter, joined in the center by a spring with smaller diameter.

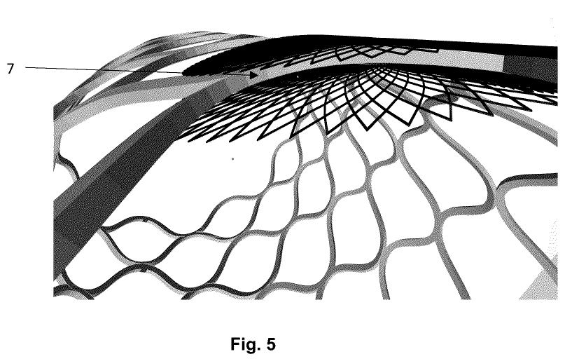

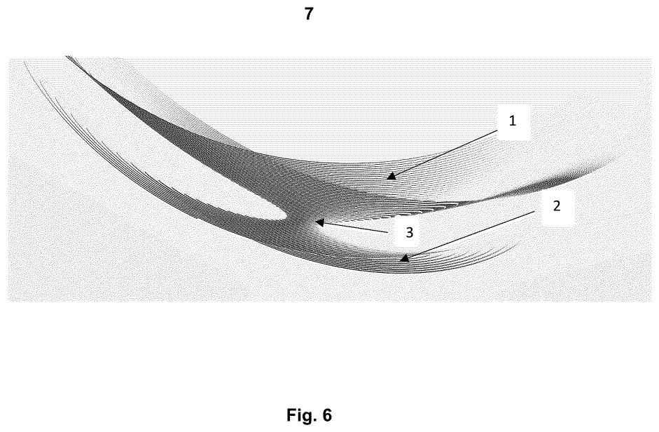

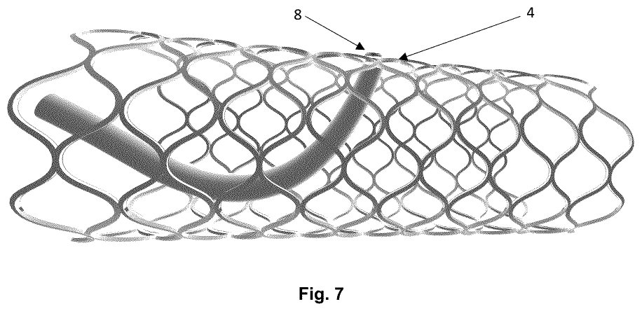

The operator begins by navigating a microcatheter 8 through the stent cell 4, as illustrated in FIG. 7. This stent cell is strategically chosen—it lies at the central position within the aneurysm's neck area. The stent itself was previously implanted, serving as a scaffold to stabilize the vessel. The distal end of the microcatheter is advanced with utmost precision. It should extend at least one millimeter distal to the plane of the aneurysm's neck. This strategic positioning ensures optimal coverage and effective deployment of the occlusive barriers. They will be positioned on either side of the stent see FIG. 1 and FIG. 5, forming a protective shield, as their central purpose is to divert blood flow into the aneurysm, reducing the risk of rupture.

Once the microcatheter is precisely positioned, the operator embarks on the delicate process of the device deployment. The goal is to navigate the filament, which will serve as the flow diverting device. As the microcatheter is retracted, the filament gradually assumes its memory shape. Initially, it forms the largest diameter loop of the distal spiral. Subsequently, smaller loops emerge, culminating in the formation of the intermediate spring (FIG. 3a). At this critical juncture, the operator must verify, employing high-resolution fluoroscopy, that the spring fully traverses the thickness of the stent struts constituting the central cell. This meticulous confirmation ensures the successful deployment of the flow-diverting mechanism. FIG. 3b, FIG. 3c. It's worth noting that the best available evidence supports using a single endoluminal device for most indications in the context of aneurysms.

Once this critical confirmation is secured, the operator continues retracting the microcatheter, now within the lumen of the parent vessel. The loops of the proximal spiral gradually reconstitute, starting from the smallest diameter and most central loop, and progressing to the largest diameter and most peripheral one. As the microcatheter retraction proceeds, a significant milestone emerges: the mark that designates the device-micro guidewire pusher junction becomes visible. At this juncture, the operator can confidently proceed with the filament detachment.

Observing the device closely, the operator notes its precise configuration:
The distal spiral nestles against the aneurysmal face of the stent.
The intermediate spring occupies the channel formed by the struts of the chosen cell within the stent.
The proximal spiral aligns with the vascular face of the stent. See FIG. 1.

This meticulous set ensures the successful deployment of the flow-diverting mechanism.

In relation to the dimensions of each of the components of the device, the specifications are crucial for achieving optimal outcomes. Here's how the distal spiral, which is positioned on the aneurysmal face of the stent, should have a diameter between 50% and 90% of the aneurysm neck's diameter. By maintaining this range, we ensure that the distal spiral does not press against the aneurysm walls. The intermediate spring lies within the stent cell and serves as a bridge between the distal and proximal spirals. Its diameter should fall between 50% and 90% of the diameter of the stent cell it crosses. This sizing allows the intermediate spring to slide comfortably without rubbing against the stent struts. Smooth movement minimizes friction and ensures effective deployment. The proximal spiral diameter, situated closest to the aneurysm neck, should have a diameter between 90% and 150% of the neck's diameter, as by occupying most or nearly the entire area of the aneurysm neck, the proximal spiral provides effective coverage. This helps prevent blood flow into the aneurysm and promotes successful flow diversion.

Regarding the effects of the device as a flow diverter restricted to the aneurysm neck, it should be highlighted the fact that achieving the specified spiral dimensions and ensuring their appropriate placement within the aneurysm neck significantly impacts the aneurysm's fate. The delicate balance between flow modification and thrombosis sets the stage for successful treatment. When the spirals occupy a substantial portion of the aneurysm neck, they influence the blood flow dynamics. By doing so, they cause a reduction in the inflow jet velocity. This altered flow pattern is beneficial because it decreases the impact force on the aneurysm wall. The modified flow dynamics, resulting from the spirals' presence, can lead to aneurysm involution, that is, the gradual shrinking or collapse of the aneurysm over time. Additionally, the reduced inflow jet velocity promotes thrombosis (clot formation) within the aneurysm sac. Thrombosis further contributes to the stabilization and healing of the aneurysm.

Over subsequent weeks or months, the aneurysm's response to the altered flow conditions becomes evident. The combination of reduced inflow forces and potential thrombosis enhances the chances of long-term success. Regular follow-up imaging helps monitor the aneurysm's evolution and assess its stability.

The primary purpose of this device is to redirect blood flow specifically within the aneurysm neck. By doing so, it prevents excessive blood flow into the aneurysm sac, reducing the risk of rupture. The device achieves flow diversion without compromising the integrity of the parent vessel (the artery from which the aneurysm arises). This preservation is crucial to maintain overall blood supply and prevent damage to the vessel. Unlike some other treatments, this approach avoids implanting foreign bodies directly into the aneurysm sac. By doing so, it minimizes the risk of complications associated with foreign material within the aneurysm. The benefits extend to reducing several risks:
Parent vessel damage: by respecting the parent vessel, the risk of vessel injury during the procedure is minimized.
Arterial occlusion: the device avoids blocking other arteries related to the aneurysm, ensuring continued blood flow.
Lesion wall manipulation: manipulating the aneurysm wall can lead to complications; this approach minimizes such manipulation.
Intraoperative rupture prevention: the most dreaded complication during aneurysm intervention is intraoperative rupture of the lesion.

In summary, this device strikes a delicate balance between effective flow diversion and minimizing risks. Its design and application aim to achieve successful outcomes while prioritizing patient safety.

The pusher wire allows the device to be navigated endovascularly inside the microcatheter to reach the focus of the vascular lesion, slightly crossing a cell of a previously implanted stent. The guidewire is manufactured as a metal wire with a diameter between 0.014 and 0.016 inches (0.36 and 0.041 mm) and a length between 160 and 200 centimeters. Its proximal end is in the hands of the operator; the guidewire contacts the proximal end of the filament. In this way, the adequate working distance between the two barriers is achieved to facilitate the maneuvers by the operator, Once the union guidewire-filament is disintegrated by any of the mechanisms for detachment of endovascular implants known in the art, whether hydraulic, thermal, mechanical or electrolytic, the pusher guidewire is released from its contact with the device and the intermediate spring takes its final shape and length, allowing the two barriers, distal and proximal, to approach each other in close proximity and almost adjoining, leaving just enough space between them for the struts of a stent cell that separates them to maintain their position. One or more connectors may be used to attach the device to the navigation or supply pusher wire in such a way that its release can be caused in a controlled manner.

The type of catheter used in the endovascular transport and navigation for the present invention can have any dimension that allows it to reach through the body vasculature to the aneurysmal lesion and reach its final position, with the following range of dimensions of its external diameter: from 1.5 French (0.019 inches—0.5 mm), up to 3 French (0.039 inches—1 mm). Most often a 2 French catheter (0.026 inch—0.67 mm) will be used.

The method by which the device described in the present invention achieves the effect of occlusion or isolation from the circulation of an aneurysm, is explained below:
The operator uses a set of elements designed to couple with a stent. These elements serve as an occluding mechanism to address the aneurysm.
The device includes two barriers: the distal 2 and proximal 1 barriers, joined by an intermediate spring. The entire device is packed inside a microcatheter using endovascular techniques. The microcatheter is advanced until it is positioned in front of the aneurysm neck. The distal end of the catheter aligns with the struts of a stent cell centrally located in front of the aneurysm neck (in various planes: axial, coronal, longitudinal, and azimuthal). FIG. 7.
Deployment Sequence: Under fluoroscopy guidance, the operator advances the microcatheter to the plane of the lesion. The microcatheter is then slowly retracted, exposing the distal end of the device. The distal spiral is deployed on the aneurysmal face of the neck. The microcatheter is retracted further to expose the intermediate spring. The spring is deployed within the selected stent cell, completely crossing the channel formed by the stent struts.
Proximal barrier formation: as the operator continues retracting the microcatheter, the proximal spiral is formed. This spiral covers the area of the aneurysm neck on the vascular side of the stent (opposite to the aneurysm neck).
Release and targeted coverage: the pusher wire detachment system is activated, releasing the device. The device remains within the patient's vascular segment, covering the predefined area of the previously implanted stent. Importantly, the operator achieves this without directly manipulating the lesion itself or affecting the aneurysm wall.

In summary, this meticulous procedure allows the doctor who acts as an operator to precisely position the device, cover the targeted stent area, and achieve effective flow diversion without disturbing either the aneurysm or the parent vessel.

What is claimed is:

1. A device for repairing aneurysms, the device comprising:
a. a filamentous material adapted for insertion into a previously implanted stent within an aneurysm site,
b. said filamentous material made of a biocompatible material with shape memory,
c. said filamentous material adopting, once deployed, the shape of a double spiral joined by a spring,
d. means for intraoperative endoluminal coupling of the filamentous material to said pre-implanted stent, comprising coupling elements configured to securely attach said filamentous material to said stent,
e. the first deployed spiral of said filamentous material being the distal segment of said device, wherein said distal segment comprises a plurality of loops whose conformation begins with the most peripheral loop, followed by a plurality of loops being deployed in the same plane, with progressively smaller diameter until reaching the central loop, that continues without interruption with the loops of said spring,
f. said spring being the intermediate segment, said intermediate segment being conformed by a plurality of loops with the same diameter forming a tubular shape,
g. the second deployed spiral being the continuation of the proximal extreme of said spring, conforming the proximal segment of said device, having said proximal segment a plurality of loops whose conformation begins with the smaller and most central loop, being followed by a plurality of loops that are deployed in the same plane, with progressively greater diameter until reaching the most peripheral loop,
h. wherein said proximal component having at the most proximal extreme a detachment mechanism.

2. The device for repairing aneurysms of claim 1, wherein the diameter of the outermost loop of said distal component of said device corresponds to between 50 and 90% of the diameter of said neck of said aneurysm.

3. The device for repairing aneurysms of claim 1, wherein the diameter of said loops of said intermediate spring of said device corresponds to between 50 and 90% of the diameter of said cell of said stent.

4. The device for repairing aneurysms of claim 1, wherein the diameter of the outermost loop of said proximal component of said device corresponds to between 100 and 150% of the diameter of said neck of said aneurysm.

5. The device for repairing aneurysms of claim 1, wherein said device exerts a flow diversion effect by occupying between 60 and 100% of the area of said stent cells where said proximal and distal components are deployed.

6. The device for repairing aneurysms of claim 1, wherein said distal component of said device being deployed parallel to the longitudinal axis and in the aneurysmal side of said previously implanted stent.

7. The device for repairing aneurysms of claim 1, wherein said spring of said device being deployed along a cell of said previously implanted stent, said cell located approximately in the center of the neck of said aneurysm.

8. The device for repairing aneurysms of claim 1, wherein said proximal component of said device being deployed parallel to the longitudinal axis and in the vascular side of said previously implanted stent.

9. The device for repairing aneurysms of claim 1, wherein said distal component being the distal barrier, said proximal component being the proximal barrier, said two barriers, distal and proximal, conforming a flow diverter obstacle in the aneurysm neck area.

10. A method of placing a device for repairing aneurysms, the method comprising the steps of:
a. providing a filamentous material adapted for insertion into a previously implanted stent within an aneurysm site,
b. providing said filamentous material made of a biocompatible material with shape memory,
c. providing said filamentous material adopting, once deployed, the shape of a double spiral joined by a spring,
d. providing means for intraoperative endoluminal coupling of said filamentous material to said pre-implanted stent, comprising coupling elements configured to securely attach said filamentous material to said stent,
e. providing the first deployed spiral of said filamentous material being the distal segment of said device, wherein said distal segment comprises a plurality of loops whose conformation begins with the most peripheral loop, followed by a plurality of loops being deployed in the same plane, with progressively smaller diameter until reaching the central loop, that continues without interruption with the loops of said spring,
f. providing said spring being the intermediate segment, said intermediate segment being conformed by a plurality of loops with the same diameter forming a tubular shape,
g. providing the second deployed spiral being the continuation of the proximal extreme of said spring, conforming the proximal segment of said device, having said proximal segment a plurality of loops whose conformation begins with the smaller and most central loop, being followed by a plurality of loops that are deployed in the same plane, with progressively greater diameter until reaching the most peripheral loop,
h. providing said proximal component, wherein said proximal component having at the most proximal extreme a detachment mechanism.

11. The method according to claim 10, wherein said distal component of said device occupy between 50 and 90% of the area of said neck of said aneurysm.

12. The method according to claim 10, wherein the diameter of said loops of said intermediate spring of said device corresponds to between 50 and 90% of the diameter of said cell of said stent.

13. The method according to claim 10, wherein the diameter of the outermost loop of said proximal component of said device corresponds to between 100 and 150% of the diameter of said neck of said aneurysm.

14. The method according to claim 10, wherein said device exerts a flow diversion effect by occupying between 60 and 100% of the area of said stent cells where said proximal and distal components are deployed.

15. The method according to claim 10, wherein said distal component of said device being deployed parallel to the longitudinal axis and in the aneurysmal side of said previously implanted stent.

16. The method according to claim 10, wherein said spring of said device being deployed along a cell of said previously implanted stent, said cell located approximately in the center of the neck of said aneurysm.

17. The method according to claim 10, wherein said proximal component of said device being deployed parallel to the longitudinal axis and in the vascular side of said previously implanted stent.

18. The method according to claim 10, wherein said distal component being the distal barrier, said proximal component being the proximal barrier, said two barriers, distal and proximal, conforming a flow diverter obstacle in the aneurysm neck area.
